SEQUENCE LISTING (38 sequences)

SEQ ID NO: 1 - DNA, 73 bp, Artificial Sequence
  Synthetic
ccttcgatagctcagttggtagagcggaggactctagatccttaggtcgctggttcgattccggctcgaagga

SEQ ID NO: 2 - DNA, 82 bp, Artificial Sequence
  Synthetic
ggtagcgtggccgagcggtctaaggcgctggattctagctccagtctcttcgggggcgtgggttcaaatcccaccgctgcca

SEQ ID NO: 3 - DNA, 82 bp, Artificial Sequence
  Synthetic
gacgaggtggccgagtggttaaggcgatggactctaaatccattgtgctctgcacacgtgggttcgaatcccatcctcgtcg

SEQ ID NO: 4 - DNA, 73 bp, Artificial Sequence
  Synthetic
gcctggatagctcagttggtagagcatcagactctaaatctgagggtccagggttcaagtccctgttcaggcg

SEQ ID NO: 5 - DNA, 72 bp, Artificial Sequence
  Synthetic
tcccacatggtctagcggttaggattcctggttctaacccaggcggcccgggttcgactcccggtgtgggaa

SEQ ID NO: 6 - DNA, 72 bp, Artificial Sequence
  Synthetic
ggccccatggtgtaatggttagcactctggactctaaatccagcgatccgagttcaaatctcggtgggacct

SEQ ID NO: 7 - DNA, 72 bp, Artificial Sequence
  Synthetic
ggcctcgtggcgcaacggtagcgcgtctgactctagatcagaaggttgcgtgttcaaatcacgtcggggtca

SEQ ID NO: 8 - DNA, 72 bp, Artificial Sequence
  Synthetic
tcccacatggtctagcggttaggattcctggttttaacccaggcggcccgggttcgactcccggtgtgggaa

SEQ ID NO: 9 - DNA, 72 bp, Artificial Sequence
  Synthetic
ggccccatggtgtaatggttagcactctggactttaaatccagcgatccgagttcaaatctcggtgggacct

SEQ ID NO: 10 - DNA, 73 bp, Artificial Sequence
  Synthetic
ccttcgatagctcagttggtagagcggaggactttagatccttaggtcgctggttcgaatccggctcgaagga

SEQ ID NO: 11 - DNA, 83 bp, Artificial Sequence
  Synthetic
gtcaggatggccgagtggtctaaggcgccagactttagttctggtctccaatggaggcgtgggttcgaatcccacttctgaca

SEQ ID NO: 12 - DNA, 82 bp, Artificial Sequence
  Synthetic
gtagtcgtggccgagtggttaaggcgatggactttaaatccattggggtttccccgcgcaggttcgaatcctgccgactacg

SEQ ID NO: 13 - DNA, 73 bp, Artificial Sequence
  Synthetic
gcccggctagctcagtcggtagagcatgggactttaaatctcagggtcgtgggttcgagccccacgttgggcg

SEQ ID NO: 14 - DNA, 72 bp, Artificial Sequence
  Synthetic
ggcctcgtggcgcaacggtagcgcgtctgacttcagatcagaaggttgcgtgttcaaatcacgtcggggtca

SEQ ID NO: 15 - DNA, 72 bp, Artificial Sequence
  Synthetic
gcgttggtggtatagtggttagcatagctgccttcaaagcagttgacccgggttcgattcccggccaacgca

SEQ ID NO: 16 - DNA, 73 bp, Artificial Sequence
  Synthetic
gggccagtggcgcaatggataacgcgtctgacttcagatcagaagattctaggttcgactcctggctggctcg

SEQ ID NO: 17 - DNA, 72 bp, Artificial Sequence
  Synthetic
gggggtatagctcaggggtagagcatttgacttcagatcaagaggtccccggttcaaatccgggtgccccct

SEQ ID NO: 18 - DNA, 83 bp, Artificial Sequence
  Synthetic
accagaatggccgagtggttaaggcgttggacttcagatccaatggattcatatccgcgtgggttcgaaccccacttctggta

SEQ ID NO: 19 - DNA, 82 bp, Artificial Sequence
  Synthetic
gtagtcgtggccgagtggttaaggcgatggacttcaaatccattggggtttccccgcgcaggttcgaatcctgtcggctacg

SEQ ID NO: 20 - RNA, 73 nt, Artificial Sequence
  Synthetic
ccuucgauagcucaguugguagagcggaggacucuagauccuuaggucgcugguucgauuccggcucgaagga

SEQ ID NO: 21 - RNA, 82 nt, Artificial Sequence
  Synthetic
gguagcguggccgagcggucuaaggcgcuggauucuagcuccagucucuucgggggcguggguucaaaucccaccgcugcca

SEQ ID NO: 22 - RNA, 82 nt, Artificial Sequence
  Synthetic
gacgagguggccgagugguuaaggcgauggacucuaaauccauugugcucugcacacguggguucgaaucccauccucgucg

SEQ ID NO: 23 - RNA, 73 nt, Artificial Sequence
  Synthetic
gccuggauagcucaguugguagagcaucagacucuaaaucugaggguccaggguucaagucccuguucaggcg

SEQ ID NO: 24 - RNA, 72 nt, Artificial Sequence
  Synthetic
ucccacauggucuagcgguuaggauuccugguucuaacccaggcggcccggguucgacucccggugugggaa

SEQ ID NO: 25 - RNA, 72 nt, Artificial Sequence
  Synthetic
ggccccaugguguaaugguuagcacucuggacucuaaauccagcgauccgaguucaaaucucggugggaccu

SEQ ID NO: 26 - RNA, 72 nt, Artificial Sequence
  Synthetic
ggccucguggcgcaacgguagcgcgucugacucuagaucagaagguugcguguucaaaucacgucgggguca

SEQ ID NO: 27 - RNA, 72 nt, Artificial Sequence
  Synthetic
ucccacauggucuagcgguuaggauuccugguuuuaacccaggcggcccggguucgacucccggugugggaa

SEQ ID NO: 28 - RNA, 72 nt, Artificial Sequence
  Synthetic
ggccccaugguguaaugguuagcacucuggacuuuaaauccagcgauccgaguucaaaucucggugggaccu

SEQ ID NO: 29 - RNA, 73 nt, Artificial Sequence
  Synthetic
ccuucgauagcucaguugguagagcggaggacuuuagauccuuaggucgcugguucgaauccggcucgaagga

SEQ ID NO: 30 - RNA, 83 nt, Artificial Sequence
  Synthetic
gucaggauggccgaguggucuaaggcgccagacuuuaguucuggucuccaauggaggcguggguucgaaucccacuucugaca

SEQ ID NO: 31 - RNA, 82 nt, Artificial Sequence
  Synthetic
guagucguggccgagugguuaaggcgauggacuuuaaauccauugggguuuccccgcgcagguucgaauccugccgacuacg

SEQ ID NO: 32 - RNA, 73 nt, Artificial Sequence
  Synthetic
gcccggcuagcucagucgguagagcaugggacuuuaaaucucagggucguggguucgagccccacguugggcg

SEQ ID NO: 33 - RNA, 72 nt, Artificial Sequence
  Synthetic
ggccucguggcgcaacgguagcgcgucugacuucagaucagaagguugcguguucaaaucacgucgggguca

SEQ ID NO: 34 - RNA, 72 nt, Artificial Sequence
  Synthetic
gcguuggugguauagugguuagcauagcugccuucaaagcaguugacccggguucgauucccggccaacgca

SEQ ID NO: 35 - RNA, 73 nt, Artificial Sequence
  Synthetic
gggccaguggcgcaauggauaacgcgucugacuucagaucagaagauucuagguucgacuccuggcuggcucg

SEQ ID NO: 36 - RNA, 72 nt, Artificial Sequence
  Synthetic
ggggguauagcucagggguagagcauuugacuucagaucaagagguccccgguucaaauccgggugcccccu

SEQ ID NO: 37 - RNA, 83 nt, Artificial Sequence
  Synthetic
accagaauggccgagugguuaaggcguuggacuucagauccaauggauucauauccgcguggguucgaaccccacuucuggua

SEQ ID NO: 38 - RNA, 82 nt, Artificial Sequence
  Synthetic
guagucguggccgagugguuaaggcgauggacuucaaauccauugggguuuccccgcgcagguucgaauccugucggcuacg

(19) United States
(12) Patent Application Publication
(10) Pub. No.: US 2023/0089490 A1
(43) Pub. Date: Mar. 23, 2023

(54) RAAV-MEDIATED IN VIVO DELIVERY OF SUPPRESSOR TRNAS

(71) Applicant: University of Massachusetts, Boston, MA (US)

(72) Inventors: Guangping Gao, Westborough, MA (US); Craig Mendonca, Shrewsbury, MA (US); Dan Wang, Belchertown, MA (US)

(73) Assignee: University of Massachusetts, Boston, MA (US)

(21) Appl. No.: 17/767,727

(22) PCT Filed: Oct. 9, 2020
(86) PCT No.: PCT/US2020/054996

§ 371 Date: Apr. 8, 2022

Related U.S. Application Data
(60) Provisional application No. 62/990,664, filed on Mar. 17, 2020.
(60) Provisional application No. 62/913,972, filed on Oct. 11, 2019.
(60) Provisional application No. 62/914,273, filed on Oct. 11, 2019.

(51) Int. Cl. C12N 15/113; C12N 15/86; A61K 48/00; A61P 7/00; A61P 11/00; A61P 21/00

(52) U.S. Cl. CPC C12N 15/113; C12N 15/86; A61K 48/0066; A61P 7/00; A61P 11/00; A61P 21/00; C12N 2750/14143; C12N 2310/11; C12N 2310/531; C12N 2750/14171

(57) ABSTRACT

Aspects of the disclosure relate to compositions and methods for treating certain diseases associated with the presence of one or more premature stop codons in a gene, for example dominantly inherited diseases or recessively inherited diseases. In some embodiments, compositions comprise a vector (e.g., a viral vector, such as an rAAV vector) encoding one or more synthetic suppressor transfer RNAs (tRNAs) configured to read-through certain stop codons (e.g., premature stop codons). In some embodiments, the disclosure relates to methods for treating Hurler syndrome comprising administering such vectors to a subject.

### RELATED APPLICATIONS

This application is a national stage filing under 35 U.S.C. § 371 of international PCT application PCT/US2020/054996, filed Oct. 9, 2020, which claims priority under 35 U.S.C. § 119(e) to U.S. provisional patent application, U.S. Ser. No. 62/913,972, filed Oct. 11, 2019, U.S. provisional patent application, U.S. Ser. No. 62/914,273, filed Oct. 11, 2019, and U.S. provisional patent application, U.S. Ser. No. 62/990,664, filed Mar. 17, 2020, the entire contents of each of which are incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Apr. 7, 2022, is named U012070130US03-SEQ-KZM and is 10313 bytes in size.

### BACKGROUND

Genetic diseases are caused by a variety of mutations and changes to the genome. One such mutation is the introduction of a stop codon before the end of a gene, known as a premature termination codon (PTC). Since the termination codon stops translation before a full-length protein is produced, the protein may not be as effective, or it will be degraded following translation termination. This can result in a disease phenotype. PTCs have been observed to be associated with about 10-15% of genetic diseases.

### SUMMARY

Aspects of the disclosure relate to compositions and methods for treating certain diseases, for example dominantly inherited diseases, or autosomal recessive diseases such as mucopolysaccharidoses (e.g., Hurler syndrome), which are characterized by the presence of one or more nonsense mutations. The disclosure is based, in part, on compositions comprising a vector (e.g., a viral vector, such as an rAAV vector, lentiviral vector, etc.) encoding one or more synthetic suppressor transfer RNAs (tRNAs) configured to read-through certain stop codons (e.g., premature stop codons). In some embodiments, administration of synthetic suppressor tRNAs (or vectors encoding such tRNAs) to a cell or subject (e.g., a mammalian subject) results in restored (e.g., increased relative to a control) protein activity in the subject for longer periods of time than previously described PTC read-through technologies. In some embodiments, the disclosure relates to methods for treating Hurler syndrome comprising administering such vectors to a subject.

Accordingly, in some aspects, the disclosure relates to a method for treating a subject having a dominantly inherited disease characterized by one or more nonsense mutations, the method comprising administering to the subject a therapeutically effective amount of a synthetic suppressor transfer RNA (tRNA), wherein the synthetic suppressor tRNA comprises an anticodon region configured to recognize the one or more nonsense mutations in a gene of the subject.

In some aspects, the disclosure relates to a method for treating a subject having a recessively inherited disease characterized by one or more nonsense mutations, the method comprising administering to the subject a therapeutically effective amount of a synthetic suppressor transfer RNA (tRNA), wherein the synthetic suppressor tRNA comprises an anticodon region configured to recognize the one or more nonsense mutation in a gene of the subject.

In some aspects, the disclosure relates to a method for treating a subject having a mucopolysaccharide (MPS) storage disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

In some embodiments, a nonsense mutation encodes a UAG, UAA, or UGA codon. In some embodiments, an anticodon region comprises a near-cognate sense codon. In some embodiments, a near-cognate sense codon is selected from the group consisting of AAG, GAG, CAG, UGG, UCG, UUG, UAC, and UAU.

In some embodiments, a synthetic suppressor tRNA is charged with an amino acid selected from the group consisting of Lysine, Glutamic acid, Glutamine, Tryptophan, Serine, Leucine, and Tyrosine.

In some embodiments, a synthetic suppressor tRNA is encoded by an expression construct comprising a nucleic acid sequence selected from the group consisting of SEQ ID NOs: 1-19. In some embodiments, a synthetic suppressor tRNA comprises the sequence set forth in any one of SEQ ID NOs: 20-38.

In some embodiments, a nucleic acid sequence is operably linked to a promoter. In some embodiments, a promoter is an RNA polymerase III promoter. In some embodiments, a promoter is a U6 promoter.

In some embodiments, an expression construct is flanked by viral terminal repeat sequences. In some embodiments, viral terminal repeat sequences are adeno-associated virus (AAV) inverted terminal repeats (ITRs).

In some embodiments, a synthetic suppressor tRNA is encoded by a viral vector, optionally an rAAV vector or a lentiviral vector. In some embodiments, an rAAV vector is encapsidated by AAV9 capsid proteins.

In some embodiments, a MPS storage disease is Hurler syndrome (MPS I), Hurler-Scheie syndrome, Scheie syndrome, Hunter syndrome (MPS II), Sanfilippo syndrome A (MPS IIIA), Sanfilippo syndrome B (MPS IIIB), Sanfilippo syndrome C (MPS IIIC), Sanfilippo syndrome D (MPS IIID), Morquiro syndrome A (MPS IVA), Morquiro syndrome B (MPS IVB), Maroteaux—Lamy syndrome (MPS VI), Sly syndrome (MPS VII), or Natowicz syndrome (MPS IX).

In some embodiments, a gene is selected from IDUA, IDS, SGSH, NAGLU, HGSNAT, GNS, GALNS, ARSB, GUSB, and HYAL1.

In some embodiments, a synthetic suppressor transfer RNA (tRNA) is administered to a subject systemically. In some embodiments, the systemic administration is intravenous injection.

In some embodiments, administration of a synthetic suppressor transfer RNA (tRNA) results in read-through of the nonsense mutation in the gene of the subject. In some embodiments, a therapeutically effective amount results in an activity level of a protein expressed from the gene that is at least 0.5% of the activity level of a wild-type protein expressed by the gene.

In some embodiments, a subject is a mammal. In some embodiments, a subject is a human.

In some aspects, the disclosure relates to a method of increasing iduronidase activity in a cell, the method comprising administering to the cell a therapeutically effective amount of a synthetic suppressor transfer RNA (tRNA), wherein the synthetic suppressor tRNA comprises an anticodon region configured to recognize a nonsense mutation in an IDUA gene of the cell.

In some embodiments, an anticodon region comprises a near-cognate sense codon. In some embodiments, a near-cognate sense codon is selected from the group consisting of AAG, GAG, CAG, UGG, UCG, UUG, UAC, and UAU.

In some embodiments, a synthetic suppressor tRNA is charged with Tyrosine.

In some embodiments, the synthetic suppressor tRNA is encoded by a viral vector. In some embodiments, the viral vector is an rAAV vector. In some embodiments, the rAAV vector is encapsidated by AAV9 capsid proteins.

In some aspects, the disclosure relates to a method of extended mRNA translation modulation in a cell. In some embodiments, a method of extended mRNA translation modulation in a cell comprises delivering to the cell a viral vector encoding one or more synthetic suppressor transfer RNAs (tRNAs) configured to read-through certain stop codons of mRNAs in the cell. In some embodiments, the viral vector is a rAAV vector. In some embodiments, the viral vector is a lentiviral vector. In some embodiments, the one or more synthetic suppressor tRNAs induce fewer off-target readthroughs in the cell than an appropriate control cell to which is delivered aminoglycoside geneticin (G418) to stimulate read-through of the certain stop codons in the control cell.

### BRIEF DESCRIPTION OF DRAWINGS

FIG. 1 shows a schematic depicting design of synthetic suppressor tRNAs that recognize the UAG codon and read-through a GFP reporter with a premature termination codon. Lys (Lysine), Glu (Glutamic acid), Gln (Glutamine), Trp (Tryptophan), Ser (Serine), Leu (Leucine), Tyr (Tyrosine).

FIG. 2 shows representative data for co-transfection of the synthetic suppressor tRNA constructs from FIG. 1 into HEK293 cells with the Y39X GFP reporter plasmid. Efficient suppression was observed for the Serine (Ser), Tyrosine (Tyr), and Glutamine (Gln) tRNAs.

FIG. 3 shows representative data from a dual luciferase reporter system used to quantitatively measure the effectiveness of each screened synthetic suppressor tRNA. The three most effective tRNAs were Serine (Ser), Tyrosine (Tyr), and Glutamine (Gln).

FIG. 4 shows representative data from injection of a mouse model of Hurler Syndrome (e.g., an Iduronidase (IDUA) deficiency) with rAAV9 containing a single synthetic suppressor tRNA, performed at about 6 weeks. The Tyr vector had IDUA activity restoration for each injected mouse.

FIG. 5 shows representative data for four synthetic suppressor tRNAs packaged into lentivirus, which were used to infect Hurler Patient Fibroblasts. The Tyrosine tRNA was most effective at restoring IDUA activity.

FIG. 6 shows representative data for an RNA oligonucleotide of the Tyr tRNA that was transfected with the GFP reporter and compared to the U6-driven Tyr plasmid. Faint GFP expression can be seen from this synthetic Tyr suppressor tRNA.

FIG. 7 shows representative data for synthetic suppressor tRNAs that were used to co-transfect HEK293 cells with a mIdua-W392X premature termination codon (PTC) cDNA. All tested tRNAs were able to rescue the mIdua-W392X PTC, leading to full-length protein synthesis.

FIGS. 8A-8B show representative data from injections of mouse models of Hurler Syndrome (e.g., Iduronidase (IDUA) deficiency) with three different rAAV9 constructs containing 1, 2, or 3 copies of a synthetic suppressor tRNA^{tyr}. FIG. 8A shows a depiction of the experimental procedure. FIG. 8B shows the impact of the administered rAAV9 constructs on IDUA activity in liver and heart tissues of the mice ten weeks after injection.

FIGS. 9A-9B show that synthetic suppressor tRNA^{Tyr} induce fewer off-target readthroughs (FIG. 9A) while providing comparable induction of IDUA activity (FIG. 9B) in Hurler human patient fibroblasts, compared to G418.

FIGS. 10A-10B show that synthetic suppressor tRNA^{Tyr} cause less perturbation on translation elongation than G418. FIG. 10A shows the codon occupancy resulting from treatment with synthetic suppressor tRNA^{Tyr} compared to a negative control (no treatment). FIG. 10B shows the codon occupancy resulting from treatment with G418 compared to a negative control (no treatment).

### DETAILED DESCRIPTION

Aspects of the disclosure relate to compositions and methods for treating certain diseases, for example dominantly inherited diseases, recessively inherited diseases, or diseases associated with haploinsufficency or a dominant negative phenotype. In some embodiments, the diseases are related to one or more nonsense mutations in a gene of a subject that result in the presence of one or more premature termination codons in the gene. The disclosure is based, in part, on compositions comprising one or more synthetic suppressor transfer RNAs (tRNAs) configured to read-through certain stop codons (e.g., premature termination codons, “PTC”). In some embodiments, the one or more synthetic suppressor tRNAs are encoded by a vector, for example a viral vector (e.g., rAAV vector, lentiviral vector, etc.).

The disclosure is based, in part, on the recognition that AAV-based delivery of synthetic suppressor tRNAs facilitates treatment of diseases where gene replacement by AAV-based delivery is limited due to the size of such genes exceeding the packaging capacity (˜4.5-5.5 kb) of AAV genomes. Examples of genes that exceed the packaging capacity of a single rAAV genome include but are not limited to dystrophin (DMD, associated with Duchene muscular dystrophy), dysferlin (DYSF, associated with Miyoshi myopathy, Limb-girdle dystrophy, and distal myopathy), Cystic fibrosis transmembrane conductance regulator (CFTR, associated with cystic fibrosis), myosin VIIA (MYO7A, associated with Usher syndrome), ATP-binding cassette, sub-family A (ABC1), member 4 (ABCA4, associated with Stargardt disease), and Factor VIII (FVIII, associated with hemophilia A). In some embodiments, each of the foregoing diseases comprises a nonsense mutation that results in a premature stop codon (PTC). In some embodiments, the disclosure relates to delivery of a synthetic suppressor tRNA described herein to restore protein function and/or activity in a gene having a nonsense mutation that exceeds the packaging capacity of a single rAAV genome.

The disclosure is based, in part, on the recognition that AAV-based delivery of synthetic suppressor tRNAs facilitates treatment of diseases associated with genes that require tight expression regulation (e.g., therapeutic transgenes that require tight expression regulation). Examples of genes that require tight expression regulation include MECP2 (mutations can lead to, e.g., neurodevelopmental disorder Rett syndrome) and CDKL5 (mutations can lead to, e.g., CDKL5 deficiency disorder). In some embodiments, read-through therapies described by the specification (e.g., synthetic suppressor tRNAs of the disclosure), are advantageous because by targeting endogenous mRNA, read-through therapy reduces the possibility of over-expression of the target gene (e.g., MECP2 and CDKL5) and reduces the possibility of undesirable expression in tissue and/or cell types that do not typically express the target gene.

As described further in the Examples section, administration of synthetic suppressor tRNA compositions described herein resulted in a therapeutically relevant restoration of protein activity in subjects having certain dominantly inherited diseases or recessively inherited diseases characterized by loss of protein expression or activity (e.g., due to the presence of one or more premature stop codons).

In some embodiments, the disclosure provides a method for treating a subject having a dominantly inherited disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

In some embodiments, the disclosure provides a method for treating a subject having a recessively inherited disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject

### Dominantly Inherited Diseases

Aspects of the disclosure relate to compositions and methods for treating a subject that has a dominantly inherited disease. As used herein, a “dominantly inherited disease” refers to a disease caused by a mutation in one allele of a gene of a subject that causes altered protein expression or activity in the subject. A dominantly inherited disease may cause a loss of protein function or activity (e.g., haploinsufficency), expression of a dominant negative gene product (e.g., a dominant negative protein). In some embodiments, a loss of protein function or activity is caused by a nonsense mutation (e.g., a mutation that introduces one or more premature stop codons) in the gene. Examples of dominantly inherited diseases include but are not limited to Huntington's disease, retinitis pigmentosa (RP), osteogenesis imperfecta, myotonic dystrophy, spinocerebellar ataxia 3, frontotemporal dementia (FTD), neurofibranatosis (type 1 or type 2), Marfan syndrome, Von Willebrand disease, familial hypercholesterolemia, tuberous sclerosis, amyotrophic lateral sclerosis (ALS), and Autosomal Dominant Polycystic Kidney Disease (ADPKD). Dominantly inherited diseases, in some embodiments, are caused by one or more mutations in a protein encoded by a gene, for example HTT, RP1, RP2, COL1A1, COL1A2, DMPK, NF1, FBN1, VWF, TSC1, TSC2, SOD1, PKD1, PKD2, etc.

Recessively Inherited Diseases

Aspects of the disclosure relate to compositions and methods for treating a subject that has a recessively inherited disease. As used herein, a “recessively inherited disease” refers to a disease caused by a mutation or mutations in both alleles of a gene of a subject that causes altered protein expression or activity in the subject. A recessively inherited disease may cause a loss of protein function or activity. In some embodiments, a loss of protein function or activity is caused by a nonsense mutation (e.g., a mutation that introduces one or more premature stop codons) in both alleles of the gene. Examples of recessively inherited diseases include but are not limited to cystic fibrosis (CF), Fanconi anemia, Pyruvate Dehydrogenase Deficiency, Pompe's disease, Gaucher's disease, phenylketonuria (PKU), and maple syrup urine disease (MSUD). Recessively inherited diseases, in some embodiments, are caused by one or more mutations in a protein encoded by a gene, for example, CFTR, FANCA, PDHA1, GAA, GBA1, PAH, BCKDHA, etc.

### Mucopolysaccharide (MPS) Storage Diseases

Aspects of the disclosure relate to compositions and methods for treating a subject that has a mucopolysaccharide storage disease. As used herein, a “mucopolysaccharide (MPS) storage disease” refers to a metabolic disorder caused by the absence or dysfunction (e.g., reduction in protein activity) of one or more lysosomal enzymes that metabolize glycosaminoglycans. Generally, MPS storage disease are characterized by certain physical symptoms, neurological and cognitive impairment, and impaired motor function.

A subject “having a MPS storage disease” generally refers to a subject that is characterized by one or more signs or symptoms of a MPS storage disease, for example cognitive impairment, neuronal damage, neuropathy, healing loss, corneal clouding, impaired motor function, hydrocephalus, coarse facial features, dwarfism, dysplasia, hepatomegaly, splenomegaly, recurrent respiratory infections, or heart disease. Examples of MPS storage diseases include but are not limited to Hurler syndrome (MPS I), Hurler-Scheie syndrome, Scheie syndrome, Hunter syndrome (MPS II), Sanfilippo syndrome A (MPS IIIA), Sanfilippo syndrome B (MPS IIIB), Sanfilippo syndrome C (MPS IIIC), Sanfilippo syndrome D (MPS IIID), Morquiro syndrome A (MPS IVA), Morquiro syndrome B (MPS IVB), Maroteaux-Lamy syndrome (MPS VI), Sly syndrome (MPS VII), or Natowicz syndrome (MPS IX).

MPS storage diseases are typically caused by one or more mutations in an enzyme involved in glycosaminoglycan metabolism, for example IDUA, IDS, SGSH, NAGLU, HGSNAT, GNS, GALNS, ARSB, GUSB, or HYAL1. Examples of mutations in IDUA include but are not limited to Q70X (as used herein, “X” denotes an amino acid resulting in generation of a PCT), W402X, Y618X, and R628X. Examples of mutations in IDS include but are not limited to R8X, C84X, E245X, and Y466X. Examples of mutations in SGSH and NAGLU include but are not limited to Y40X, and those described by Yogalingam et al. Hum. Mutat. 18: 264-281, 2001. Examples of mutations in HGSNAT include those described by Fedele et al. Hum. Mutat. 28(5) 523 2007. One example of a mutation in GNS is Q390X. Examples of mutations in GALNS include but are not limited to W10X, W159X, W325X, and Q422T. Examples of mutations in ARSB include but are not limited to R315X, R327X, Q456X and Q503X. One example of a mutation in GUSB is G357X. One example of a mutation in HYAL1 is V35X. In some embodiments, a subject is characterized as having one or more of the foregoing mutations (e.g., determined as having one or more mutations by a genetic test).

Diseases Resulting from Dysregulation of Gene Product Expression

Aspects of the disclosure relate to compositions and methods for treating a subject that has a disease resulting from dysregulation of gene/protein expression (e.g., Rett Syndrome or CDKL5 deficiency disorder). As used herein, a disease resulting from dysregulation of gene/protein expression refers to diseases that are either initiated by or worsened by the aberrant regulation of the expression (e.g., resulting in different expression of the gene compared to expression in normal, healthy cells or tissue) of a certain gene or protein (e.g., because of introduction of a premature stop codon in the gene).

A subject having a disease resulting from dysregulation of gene product (e.g., protein) expression generally refers to a subject that is characterized by a genetic mutation in a gene that leads to abnormally low or high expression of the resultant mRNA transcript and/or protein. Examples of diseases resulting from poor regulation of gene/protein expression include Rett Syndrome and CDKL5 deficiency disorder. In some embodiments, mutations in MECP2 lead to abnormal expression of MECP mRNA and protein levels in a subject, resulting in Rett Syndrome. In some embodiments, mutations in CDKL5 lead to abnormal expression of CDKL5 mRNA and protein levels in a subject, resulting in CDKL5 deficiency disorder. In some aspects, the disclosure provides a method for treating a disease associated with dysregulation of a gene product, the method comprising administering to a subject in need thereof a synthetic suppressor tRNA as described herein. In some embodiments, the disease is Rett Syndrome or CDKL5 deficiency disorder.

Synthetic Suppressor tRNAs

Aspects of the disclosure relate to transfer RNAs (tRNAs). A “transfer RNA (tRNA)” is an oligoribonucleotide that is between about 70 and 90 nucleotides in length, binds to a messenger RNA (mRNA), and in doing so carries an amino acid to a ribosome whereupon the amino acid is added to a polypeptide chain. The cloverleaf structure of tRNAs typically comprises a 5′ terminal phosphate group, a 7-9 base pair acceptor stem (which contains a CCA-3′-terminal group to which the amino acid is attached), a “D loop” comprising a 4-6 base pair stem ending in a loop, a “T-loop” comprising a 4-5 base pair stem that includes a pseudouridine, and an anticodon arm comprising a 5 base pair stem ending in a loop containing an anticodon (a three nucleotide sequence that binds to a codon of mRNA). The structure of tRNAs is known and described, for example by Sharp et al. Crit. Rev. Biochem. 19:107 144 (1985).

In some embodiments, a transfer RNA is a synthetic suppressor tRNA. As used herein, a “synthetic suppressor tRNA” refers to a transfer RNA that has been configured (e.g., modified) to bind to a termination codon. Without wishing to be bound by any particular theory, in some embodiments, synthetic suppressor tRNAs described herein are configured to allow for “read-through” of a premature termination codon, thus allowing production of a functional or partially functional protein from the gene containing the mutation causing the premature termination codon.

A synthetic suppressor tRNA can bind any termination codon, for example an amber codon (UAG), an ochre codon (UAA), or an opal codon (UGA). In some embodiments, a synthetic suppressor tRNA comprises an anticodon that binds to a termination codon (e.g., UAG, UAA, UGA). In some embodiments, a synthetic suppressor tRNA comprises an anticodon region configured to recognize a nonsense mutation in a gene (e.g., a nonsense mutation that results in the gene having a premature termination codon). In some embodiments, a synthetic suppressor tRNA binds to a premature stop codon resulting from a mutation in a gene selected from IDUA, IDS, SGSH, NAGLU, HGSNAT, GNS, GALNS, ARSB, GUSB, and HYAL1. In some embodiments, a synthetic suppressor tRNA preferentially binds to a premature termination codon relative to a normal stop codon (e.g., a stop codon upstream of a polyA tail of an mRNA). Methods of producing and testing synthetic suppressor tRNAs are known, for example as described by Lueck et al. Nature Communications 10:822 2019.

An anticodon (e.g., an anticodon of a synthetic suppressor tRNA) that binds to a termination codon may be a cognate anticodon (e.g., the anticodon forms three base pairs with the termination codon) or a near-cognate anticodon (e.g., the anticodon forms two base pairs with the termination codon). Examples of near-cognate tRNAs are shown below in Table 1.

TABLE 1
Near-cognate tRNAs and their charged amino acid
Near Cognate tRNAs

| UAA | UAG | UGA |
|---|---|---|
| AAA-Lys | AAG-Lys | AGA-Arg |
| CAA-Gln | CAG-Gln | CGA-Arg |
| GAA-Glu | GAG-Glu | GGA-Gly |
| UCA-Ser | UCG-Ser | UCA-Ser |
| UUA-Leu | UGG-Trp | UUA-Leu |
| UAC-Tyr | UUG-Leu | UGC-Cys |
| UAU-Tyr | UAC-Tyr | UGG-Trp |
| | UAU-Tyr | UGU-Cys |

In some embodiments, a synthetic suppressor tRNA comprises the nucleic acid sequence set forth in any one of SEQ ID NOs: 20-38.

A synthetic suppressor tRNA may be charged with any amino acid. As used herein, a “charged” tRNA refers to a tRNA that has been chemically bonded to its cognate amino acid. In some embodiments, a tRNA is charged with a natural amino acid (e.g., Ala, Arg, Asn, Asp, Cys, Gln, Glu, Gly, His, Ile, Leu, Lys, Met, Phe, Pro, Ser, Thr, Trp, Tyr, Val). In some embodiments, a tRNA is charged with an unnatural amino acid (e.g., a non-alpha amino acid, a D-amino acid, a dehydroamino acid, selenol amino acids, etc.).

In some embodiments, binding of a synthetic suppressor tRNA described by the disclosure to a premature termination codon of an mRNA causes “read-through” of the premature stop codon by a ribosome and results in production of a full-length protein. In some embodiments, “read-through” of an mRNA causes an increase in protein level or activity in a cell or subject that ranges from about 0.1% to about 100% (e.g., an increase of about 0.1%, 0.5%, 1.0%, 2%, 5%, 10%, 25%, 50%, 75%, or 100%) relative to protein expression or activity of an mRNA that has not been contacted with the synthetic suppressor tRNA. In some embodiments, “read-through” of an mRNA causes an increase in protein level or activity of more than 100% (e.g., 200%, 500%, 1000%, etc.) in a cell or subject.

### Isolated Nucleic Acids

In some aspects, the disclosure provides isolated nucleic acids that are useful for expressing synthetic suppressor tRNAs. A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

In some embodiments, an isolated nucleic acid encodes a synthetic suppressor tRNA having the nucleic acid sequence set forth in any one of SEQ ID NOs: 20-38.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more molecules (e.g., one or more synthetic suppressor tRNAs). The transgene may also comprise a region encoding, for example, a miRNA binding site, and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In some embodiments, one or more additional nucleotide sequences are found between the transgene and the 5′ and/or 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, AAV9.hr, AAVrh8, AAVrh10, AAVrh39, AAVrh43, AAV.PHP, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAV11, AAV9.hr, AAVrh8, AAVrh10, AAVrh39, AAVrh43, AAV.PHP, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or mis sense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences (e.g., Nrl-response element, CRX-response element, RET-1, etc.); efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized. In some embodiments, an isolated nucleic acid comprises one or more non-functional “stuffer sequences”. A stuffer sequence may comprise between about 10 and about 2000 contiguous nucleotides, and generally functions to ensure proper viral vector packaging.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is a CB6 promoter. In some embodiments, a promoter is an enhanced chicken β-actin promoter. In some embodiments, a promoter is a U6 promoter. In some embodiments, a promoter is a chicken beta-actin (CBA) promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the tissue-specific promoter is a CNS-specific promoter. Examples of CNS-specific promoters include but are not limited to neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)).

Aspects of the disclosure relate to isolated nucleic acids comprising RNA polymerase III promoters. Without wishing to be bound by any particular theory, use of RNA polymerase III promoters are advantageous relative to RNA polymerase II promoters because they 1) do not compete with endogenous RNA polymerase II promoters for production of mRNAs, 2) in eukaryotic cells pol III promoters drive production of endogenous tRNAs, and 3) pol III promoters allow for high levels of suppressor tRNA expression without cellular toxicity. In some embodiments, an isolated nucleic acid comprises a transgene encoding one or more synthetic suppressor tRNAs and an RNA polymerase III promoter (e.g., U6 promoter). Non-limiting examples of pol III promoters include U6 and H1 promoter sequences.

In some embodiments, a promoter is an RNA polymerase II (pol II) promoter. Non-limiting examples of pol II promoters include T7, T3, SP6, RSV, and cytomegalovirus promoter sequences.

In some aspects, the disclosure relates to isolated nucleic acids comprising a transgene encoding one or more synthetic suppressor tRNAs, and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the nucleotide of a nucleic acid sequence encoding a synthetic suppressor tRNA, and a poly A sequence.

In some embodiments, a transgene comprises one or more (e.g., 1, 2, 3, 4, 5, or more) miRNA binding sites that de-target expression of the one or more synthetic suppressor tRNAs from non-central nervous system (CNS) cells, such as liver cells.

In some embodiments, a transgene comprises one or more (e.g., 1, 2, 3, 4, 5, or more) miRNA binding sites that de-target expression of one or more synthetic suppressor tRNAs from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell. or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152.

### Vectors

In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV7, AAV8, AAVrh8, AAV9, AAV10, AAV9.hr, AAVrh8, AAVrh10, AAVrh39, AAVrh43, and AAV.PHP. In some embodiments, an AAV capsid protein is of a serotype derived from a non-human primate, for example AAVrh8 serotype. In some embodiments, an AAV capsid protein is an evolved capsid protein or non-naturally occurring capsid protein (e.g., for example as described by Buning and Srivastava Mol Ther Methods Clin Dev. (2019) 12:248-265).

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises a coding sequence encoding one or more synthetic suppressor tRNAs. In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. In some embodiments, a vector encodes one or more synthetic suppressor tRNAs as described herein.

The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., synthetic suppressor tRNA) from a transcribed gene.

A gene therapy vector may be a viral vector (e.g., a lentiviral vector, an adeno-associated virus vector, etc.), a plasmid, a closed-ended DNA (e.g., ceDNA), etc. In some embodiments, a gene therapy vector is a viral vector. In some embodiments, an expression cassette encoding a minigene is flanked by one or more viral replication sequences, for example lentiviral long terminal repeats (LTRs) or adeno-associated virus (AAV) inverted terminal repeats (ITRS).

The foregoing methods for packaging recombinant vectors in desired AAV capsids to produce the rAAVs of the disclosure are not meant to be limiting and other suitable methods will be apparent to the skilled artisan.

### Methods

Aspects of the disclosure relate to methods for delivering one or more (e.g., 1, 2, 3, 4, 5, or more) synthetic suppressor tRNAs to a subject (e.g., a cell in a subject). In some embodiments, the subject is a human. In some embodiments, the subject is a non-human mammal. Non-limiting examples of non-human mammals are mouse, rat, cat, dog, sheep, rabbit, horse, cow, goat, pig, guinea pig, hamster, chicken, turkey, or a non-human primate.

Accordingly, in some aspects, the disclosure relates to a method for treating a subject having a mucopolysaccharide (MPS) storage disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

In some embodiments, the term “treating” refers to the application or administration of a synthetic suppressor tRNA (or a vector encoding a synthetic suppressor tRNA) to a subject (or a cell), who has a mucopolysaccharide (MPS) storage disease (e.g., Hurler syndrome, etc.), or a predisposition toward a mucopolysaccharide (MPS) storage disease, with the purpose to cure, heal, alleviate, relieve, alter, remedy, ameliorate, improve, or affect the disorder, the symptom of the disease, or the predisposition toward the mucopolysaccharidosis.

In some embodiments, “treating” refers to the application or administration of a synthetic suppressor tRNA (or a vector encoding a synthetic suppressor tRNA) to a subject (or a cell), who has a disease resulting from poor regulation of gene/protein expression (e.g., Rett Syndrome or CDKL5 deficiency disorder).

In some embodiments, administration of a synthetic suppressor tRNA causes an increase in a protein level or activity in a cell or subject that ranges from about 0.1% to about 100% (e.g., an increase of about 0.1%, 0.5%, 1.0%, 2%, 5%, 10%, 25%, 50%, 75%, or 100%) relative to protein expression or activity of an mRNA that has not been contacted with the synthetic suppressor tRNA. In some embodiments, administration of a synthetic suppressor tRNA causes an increase in protein level or activity of more than 100% (e.g., 200%, 500%, 1000%, etc.) in a cell or subject. Methods of measuring protein level or activity are known in the art. A non-limiting exemplary reference value can be a level of protein or activity of the same subject prior to receiving the synthetic suppressor tRNA.

In some embodiments, an increase in a protein level or activity (e.g., relative to a control) lasts for at least 3 weeks, 4, weeks, 5, weeks, 6 weeks, 7 weeks, 8 weeks, 9 weeks, or 10 weeks after administration of the synthetic suppressor tRNAs. In some embodiments, an increase in protein level or activity lasts for more than 10 weeks (e.g., 3 months, 4 months, 5 months, 6 months, 1 year, etc.).

Alleviating a mucopolysaccharide (MPS) storage disease or a disease resulting from dysregulation of gene product (e.g., protein) expression includes delaying the development or progression of the disease, or reducing disease severity. Alleviating the disease does not necessarily require curative results. As used therein, “delaying” the development of a disease (such as a mucopolysaccharide (MPS) storage disease, etc.) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease (e.g., mucopolysaccharide (MPS) storage disease, etc.) includes initial onset and/or recurrence.

An “effective amount” of a substance is an amount sufficient to produce a desired effect. In some embodiments, an effective amount of a synthetic suppressor tRNA (e.g., an isolated nucleic acid comprising a transgene encoding a synthetic suppressor tRNA or a vector comprising such a nucleic acid) is an amount sufficient to transfect (or infect in the context of rAAV mediated delivery) a sufficient number of target cells of a target tissue of a subject. In some embodiments, a target tissue central nervous system tissue (e.g., neural cells, glial cells, axons, dendrites, oligodendrocytes, etc.). In some embodiments, an effective amount of an isolated nucleic acid (e.g., which may be delivered via an rAAV) may be an amount sufficient to have a therapeutic benefit in a subject, e.g., to increase or supplement the expression of a gene or protein of interest (e.g., IDUA, IDS, SGSH, NAGLU, HGSNAT, GNS, GALNS, ARSB, GUSB, HYAL1, MECP2, CDKL5, etc.), to improve in the subject one or more symptoms of disease (e.g., a symptom of a mucopolysaccharide (MPS) storage disease), etc. The effective amount will depend on a variety of factors such as, for example, the species, age, weight, health of the subject, and the tissue to be targeted, and may thus vary among subject and tissue as described elsewhere in the disclosure.

Compositions (e.g., synthetic suppressor tRNAs, rAAVs, etc.) may be delivered to a subject in compositions according to any appropriate methods known in the art. The composition, preferably suspended in a physiologically compatible carrier, may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments, a host animal does not include a human.

The compositions of the disclosure may comprise an active ingredient (e.g., synthetic suppressor tRNAs, rAAVs, etc.) alone, or in combination with one or more other agents. In some embodiments, a composition comprises 1, 2, 3, 4, 5, 6, 7, 8, 9, 10, or more different rAAVs each having one or more different transgenes (e.g., transgenes encoding different synthetic suppressor tRNAs).

In some embodiments, a composition further comprises a pharmaceutically acceptable carrier. Suitable carriers may be readily selected by one of skill in the art in view of the indication for which the composition is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the active ingredient and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions (e.g., compositions comprising one or more synthetic suppressor tRNAs) are administered in sufficient amounts to transfect the cells of a desired tissue (e.g., CNS tissue) and to provide sufficient levels of gene transfer and expression without undue adverse effects. Examples of pharmaceutically acceptable routes of administration include, but are not limited to, direct delivery to the selected organ (e.g., subretinal delivery to the eye), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors.

An effective amount of an rAAV is an amount sufficient to target infect an animal, target a desired tissue. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In some embodiments, an effective amount is produced by multiple doses of an rAAV.

In some embodiments, a dose is administered to a subject no more than once per calendar day (e.g., a 24-hour period). In some embodiments, a dose is administered to a subject no more than once per 2, 3, 4, 5, 6, or 7 calendar days. In some embodiments, a dose is administered to a subject no more than once per calendar week (e.g., 7 calendar days). In some embodiments, a dose is administered to a subject no more than bi-weekly (e.g., once in a two calendar week period). In some embodiments, a dose is administered to a subject no more than once per calendar month (e.g., once in 30 calendar days). In some embodiments, a dose is administered to a subject no more than once per six calendar months. In some embodiments, a dose is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Appropriate methods for reducing aggregation of may be used, including, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, rAAVs in suitably formulated pharmaceutical compositions disclosed herein are delivered directly to target tissue, e.g., direct to CNS tissue. However, in certain circumstances it may be desirable to separately or in addition deliver the rAAV-based therapeutic constructs via another route, e.g., subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by intravitreal injection or subretinal injection.

The pharmaceutical forms suitable for injectable use include suspension-based formulations, sterile aqueous solutions or dispersions, and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a suitable sterile aqueous medium may be employed. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the disclosure relates to a kit for producing a rAAV containing a suppressor tRNA. In some embodiments, the kit further comprises instructions for producing the rAAV containing a suppressor tRNA. In some embodiments, the kit further comprises at least one container housing a recombinant AAV vector, wherein the recombinant AAV vector comprises a transgene encoding a suppressor tRNA.

In some embodiments, the disclosure relates to a kit comprising a container housing a recombinant AAV as described supra. In some embodiments, the kit further comprises a container housing a pharmaceutically acceptable carrier. For example, a kit may comprise one container housing a rAAV and a second container housing a buffer suitable for injection of the rAAV into a subject. In some embodiments, the container is a syringe.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

In some cases, the methods involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some cases, RNA from the transfected cells provides a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells may also be infected with a helper virus, such as an Adenovirus or a Herpes Virus. In a specific embodiment, the helper functions are provided by an adenovirus. The adenovirus may be a wild-type adenovirus, and may be of human or non-human origin, preferably non-human primate (NHP) origin. Similarly adenoviruses known to infect non-human animals (e.g., chimpanzees, mouse) may also be employed in the methods of the disclosure (See, e.g., U.S. Pat. No. 6,083,716). In addition to wild-type adenoviruses, recombinant viruses or non-viral vectors (e.g., plasmids, episomes, etc.) carrying the necessary helper functions may be utilized. Such recombinant viruses are known in the art and may be prepared according to published techniques. See, e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells may also be transfected with a vector (e.g., helper vector) which provides helper functions to the AAV. The vector providing helper functions may provide adenovirus functions, including, e.g., E1a, E1b, E2a, E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

In some cases, a novel isolated capsid gene can be used to construct and package recombinant AAV vectors, using methods well known in the art, to determine functional characteristics associated with the novel capsid protein encoded by the gene. For example, novel isolated capsid genes can be used to construct and package recombinant AAV (rAAV) vectors comprising a reporter gene (e.g., B-Galactosidase, GFP, Luciferase, etc.). The rAAV vector can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1

Genetic diseases are caused by a variety of mutations and changes to the genome. One such mutation is the introduction of a stop codon before the end of a gene, known as a premature termination codon (PTC). Since the termination codon stops translation before a full-length protein is produced, the protein may not be as effective, or it will be degraded following translation termination. This can result in a disease phenotype. PTCs are the cause of about 10-15% of genetic diseases. Examples of genes and diseases associated with PTCs include but are not limited to DMD (associated with Duchene muscular dystrophy), DYSF (associated with Miyoshi myopathy, Limb-girdle dystrophy, and distal myopathy), CFTR (associated with cystic fibrosis), MYO7A (associated with Usher syndrome), ABCA4 (associated with Stargardt disease), FVIII (associated with hemophilia A), HBB (associated with beta-thalassemia), SCN1A (associated with Dravet syndrome), and SMN1 (associated with spinal muscular atrophy). PTCs have also been associated with certain cancers (e.g., cancers caused by nonsense mutations in p53 or PTEN).

PTCs can be created by several mechanisms. A nonsense substitution mutation (transition or transversion) could cause a triplet that encodes for an amino acid to change to a stop codon. An insertion or deletion mutation (+/−1 bp) could also create a stop codon. However, reading through this stop codon could produce an aberrant protein that will most likely be degraded by one of the proteolytic pathways. An engineered suppressor tRNA that recognizes 4 bases (three of which are part of the stop codon) could be used to read through the stop codon as well as correct a +1 bp frameshift mutation.

Other approaches have been used to target and treat PTC mutations. For example, by using gene-editing technology (e.g., CRISPR/Cas9), a corrected sequence replaces the mutations and the full-length protein is produced. One issue with this strategy is that the transgene could be immunogenic, which would cause harm to the patient. Another issue is that there could be off-target effects in CRISPR targeting, which could result in unwanted genetic modifications. A new vector would need to be designed for each mutation or disease, which greatly hinders the development of rare disease treatments.

Another advantage of using suppressor tRNA is that, because there is no exogenous protein being produced, immunogenicity is much reduced. This is in contrast to CRISPR-Cas9 based genome editing, in which case the Cas9 proteins can be highly immunogenic.

### Example 2

This example describes compositions and methods for treating diseases associated with premature termination codons (PTC, also referred to as premature stop codons), such as Hurler syndrome (mucopolysaccharidosis (MPS) type I).

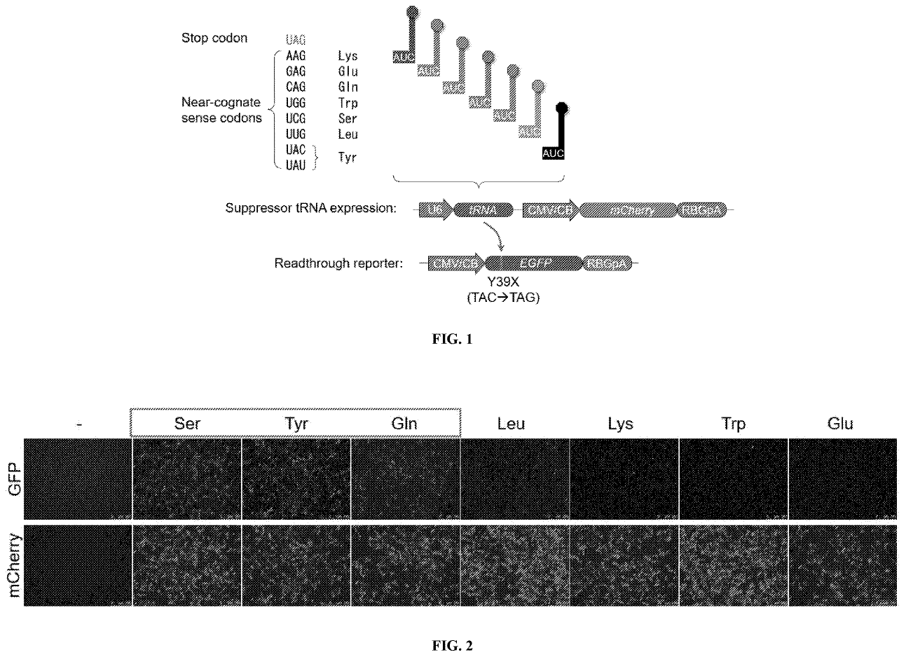

Several synthetic suppressor transfer RNAs (tRNAs) were produced. Each engineered suppressor tRNA has a 3′-AUC-5′ anticodon to recognize the PTC 5′-UAG-3′. At the normal translational termination site, there are usually multiple termination codons and other sequence elements that signal the ribosome to disengage from the mRNA and finish making the polypeptide. However, at a PTC, these other elements are not present. This allows a suppressor tRNA to recognize the PTC and insert an amino acid where the protein would normally be truncated. This strategy is exemplified in FIG. 1 using a GFP read-through reporter system. In this system, the Y39X PTC prevents GFP expression in cell culture, but the suppressor tRNA is able to read-through this mutation and continue with protein synthesis.

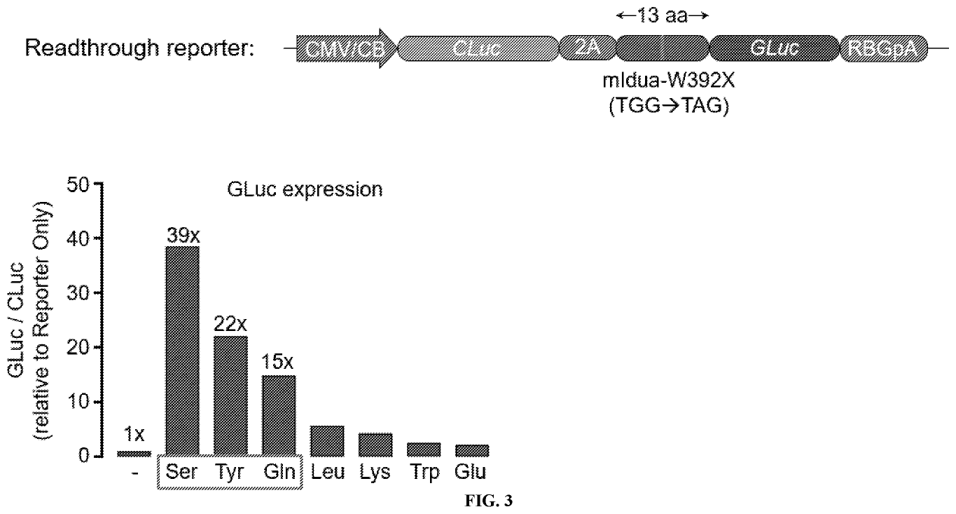

Seven suppressor tRNAs were screened using this reporter system by co-transfection into HEK293 cells, as shown in FIG. 2. Three of the tRNAs mediated efficient read-through: serine tRNA (tRNA^{Ser}), tyrosine tRNA (tRNA^{Tyr}), and glutamine tRNA (tRNA^{Gln}). The reporter plasmid also included mCherry driven by the CMV/CB promoter, which can be used as a qualitative control for transfection efficiency.

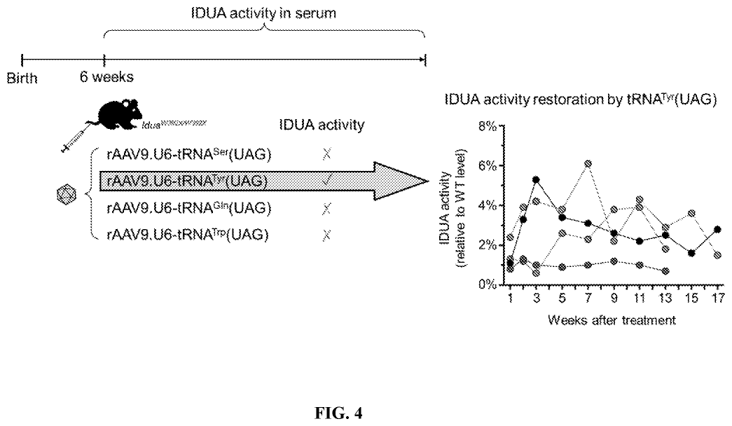

The tRNAs were also tested using a dual-luciferase system, as described in FIG. 3. Briefly, Cypridina Luciferase (Cluc) is expressed upstream and Guassia Luciferase (Gluc) is expressed downstream of a middle section, which comprises a self-cleaving 2A peptide and a mouse IDUA gene (mIDUA) W392X PTC region. In all experiments, Cluc is expressed and acts as a quantitative transfection control. Gluc is only expressed if read-through occurs, so a high Gluc to Cluc ratio indicates efficient read-through. Synthetic suppressor tRNAs charged with Serine, Tyrosine, and Glutamine had the highest ratios of Gluc/Cluc, indicating they were the most efficient at reading through this mutation.

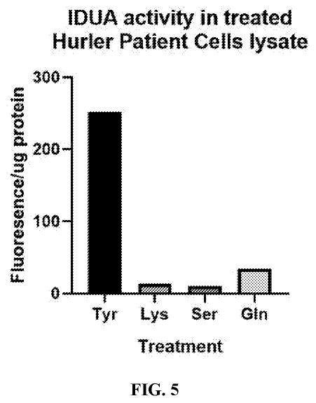

Serine, Tyrosine, Glutamine, and Tryptophan (the natural amino acid at position 392 of mIDUA) tRNAs were packaged into rAAV9 and delivered to a mouse model of MPS I (Hurler Mice). Representative data, shown in FIG. 4 indicate that the Tyrosine tRNA (tRNA^{Tyr}) demonstrated a restoration in IDUA activity in vivo. In order to reverse the Hurler phenotype, a small percentage (less than 0.5%) of IDUA activity restoration is required; treatment with the tRNA^{Tyr} rAAV far exceeded this therapeutic threshold. The rAAV9.U6-tRNA^{Tyr} treated mice were monitored for 17 weeks post-administration and maintained a clinically relevant IDUA serum level through that time point.

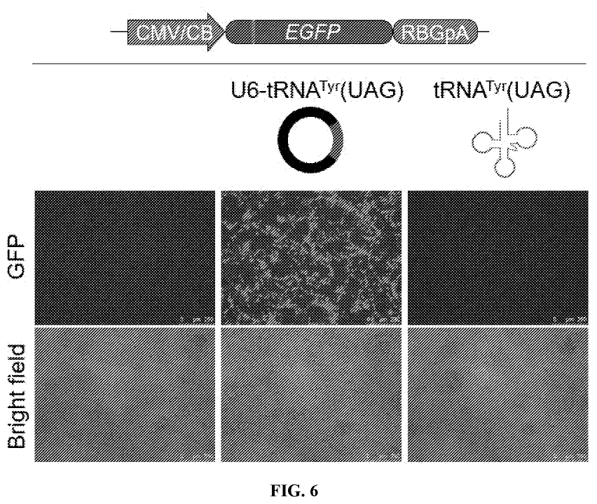

Four of the suppressor tRNAs (tRNA^{Tyr}, tRNA^{Lys}, tRNA^{Ser}, tRNA^{Gln}) were packaged into lentivirus and used to infect fibroblast cells from a Hurler Syndrome patient with homozygous IDUA-W402X mutation. These fibroblast cells have no IDUA activity due to a PTC mutation (W420X). Following lentivirus infection, the patient cells were collected, and the protein lysate was assayed for IDUA activity, normalized to the protein amount. As shown in FIG. 5, tRNA^{Tyr} was able to restore the greatest IDUA activity compared to the other tRNAs tested.

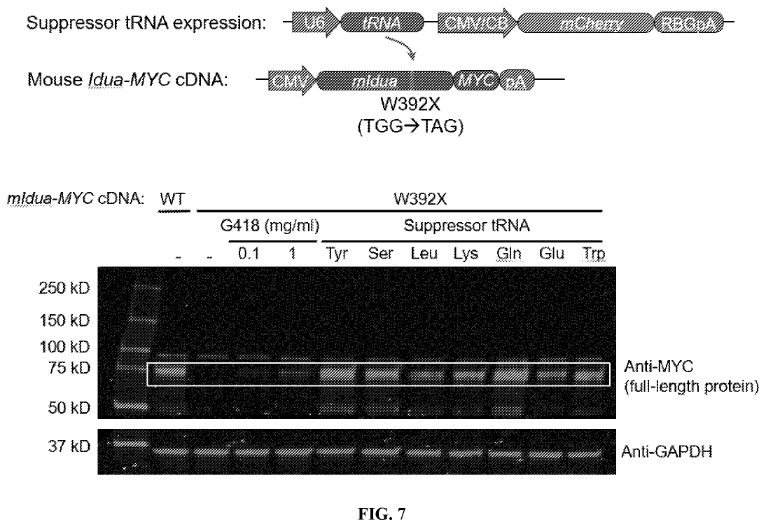

Suppressor tRNAs were also transfected directly to cell culture (e.g., not via a plasmid). Synthetic tRNAs were synthesized as RNA oligonucleotides and co-transfected with the Y39X EGFP reporter. FIG. 6 shows read-through of the GFP PTC, indicating that suppressor tRNAs may be directly delivered to a subject.

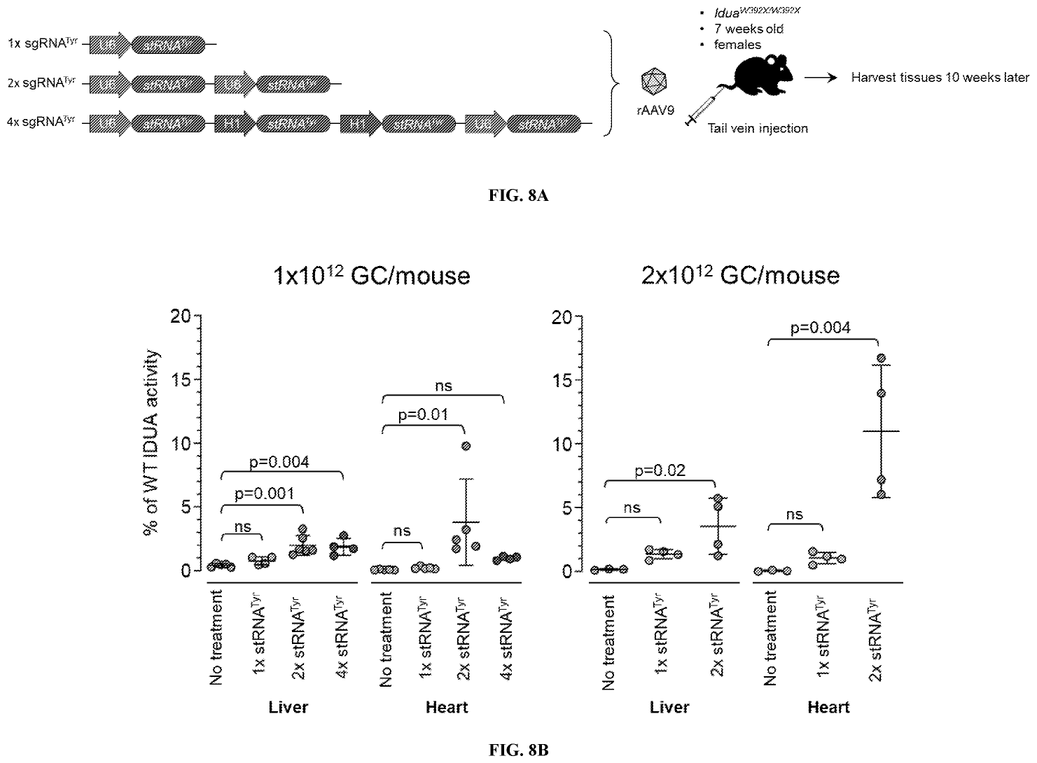

All seven of the suppressor tRNAs (tRNA^{Tyr}, tRNA^{Ser}, tRNA^{Leu}, tRNA^{Lys}, tRNA^{Gln}, tRNA^{Glu}, tRNA^{Trp}) were able to rescue a premature termination codon (PTC) from a co-transfected cDNA in HEK293 cells. A plasmid encoding one of the suppressor tRNAs and a cDNA encoding a mIdua-MYC construct (mIdua gene having a W392X premature termination codon (PTC) mutation fused to a myc gene) were co-transfected into HEK293 cells. After a 48-hour incubation period, the transfected HEK293 cells were treated with the aminoglycoside geneticin (G418), a known modulator of translational read-through. After an additional 24-hour period, the transfected HEK293 cells were harvested. A western blot analysis to test for expression of the mIdua-MYC construct was performed (FIG. 7). Cells that were treated with G418 and no suppressor tRNAs showed little-to-no expression of full-length mIdua-MYC construct. However, each of the suppressor tRNAs were able to rescue the expression of the full-length mIdua-MYC construct in cells treated with the read-through modulator G418.

### Example 3

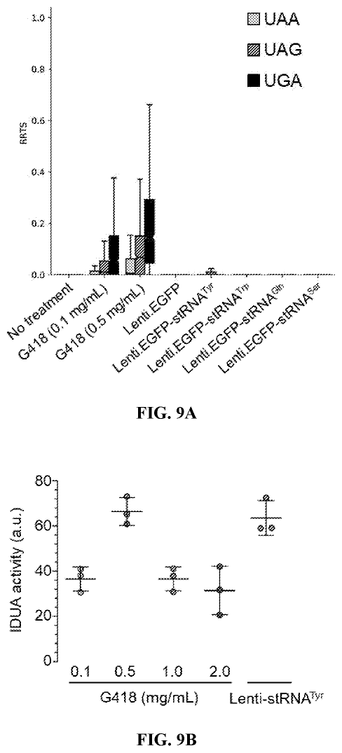

Tyrosine tRNAs were packaged into rAAV9 vectors and delivered to a mouse model of MPS I (Hurler Mice). As shown in FIG. 8A, three constructs were generated—(1) rAAV9 containing one suppressor tRNA^{Tyr} gene downstream of a U6 promoter, (2) rAAV9 containing two copies of a suppressor tRNA^{Tyr} gene downstream of a U6 promoter, and (3) rAAV9 containing four copies of a suppressor tRNA^{Tyr} gene downstream of a U6 promoter—and subsequently injected twice into the tail vein of seven-week old female mice that are homozygous for mutated IDUA gene with a premature stop codon (W392X). Mice were injected with either 1×10¹² or 2×10¹² genome copies (GC/mouse). In addition to the treatment groups, a group of mice were injected with saline solution as a negative control (‘no treatment’). Ten weeks after injection, all mice were euthanized and tissues were harvested.

Liver and heart tissues of the mice that were treated with the rAAV9 containing two copies of a suppressor tRNA^{Tyr} gene downstream of a U6 promoter and those treated with the rAAV9 containing four copies of a suppressor tRNA^{Tyr} gene downstream of a U6 promoter had statistically significant increases in the percent of wild-type (WT) IDUA activity, relative to mice treated with saline (FIG. 8B). For example, liver tissues of the mice that were treated with 2×10¹² genome copies of the rAAV9 containing two copies of a suppressor tRNA^{Tyr} gene had ˜3.5% of WT IDUA compared to ˜0% of WT IDUA in liver tissues of untreated mice. Heart tissues of the mice that were treated with 2×10¹² genome copies of the rAAV9 containing two copies of a suppressor tRNA^{Tyr} gene had ˜11% of WT IDUA compared to ˜0% of WT IDUA in liver tissues of untreated mice. P values as shown in FIG. 8B were calculated by one-way ANOVA followed by Dunnet's multiple comparisons test (ns: not significant).

### Example 4

The suppressor tRNAs of the disclosure induced fewer off-target readthroughs than the aminoglycoside geneticin (G418). Specifically, lentiviral delivery of suppressor tRNAs (tRNA^{Tyr}, tRNA^{Trp}, tRNA^{Gln}, tRNA^{Ser}) to Hurler patient fibroblasts induced fewer off-target readthroughs than G418.

A lentivirus containing one of the suppressor tRNAs (tRNA^{Tyr}, tRNA^{Trp}, tRNA^{Gln}, tRNA^{Ser}) and a gene encoding an Enhanced Green Fluorescent Protein (EGFP) construct was transfected into Hurler patient fibroblasts. Similarly, Hurler patient fibroblasts were treated with varying concentrations of G418 (0.1 mg/mL, 0.5 mg/mL, 1.0 mg/mL, or 2.0 mg/mL). After an incubation period, the fibroblasts were harvested and assessed by next-generation sequencing and for IDUA activity.

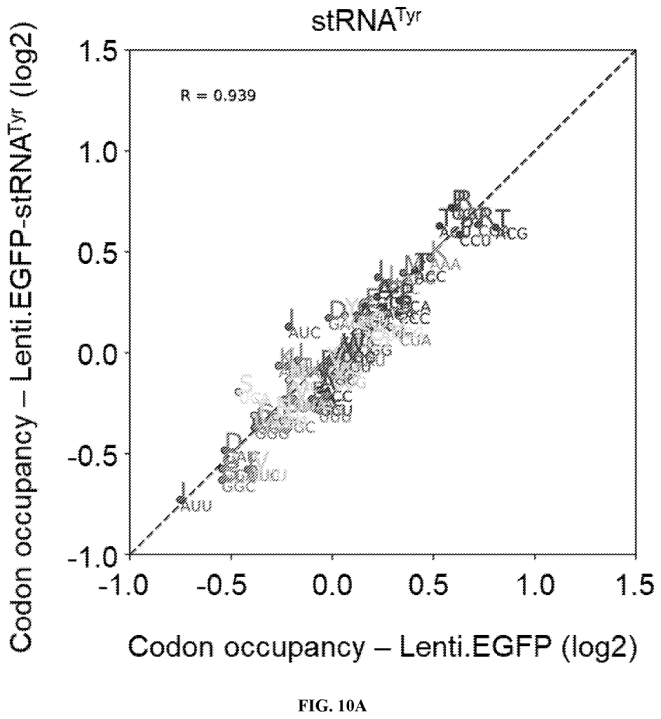

Ribosome profiling, a technique to query ribosome-protected mRNA fragments by next-generation sequencing, revealed ribosome occupancy at the 3′ untranslated region (UTR) of the transcriptome in fibroblasts that had been treated with G418, but not in fibroblasts that had been treated with lentiviral vectors expressing the suppressor tRNAs. Further, the treated cells were assessed for a ribosome readthrough score (RRTS), which represents the density of ribosomes in the 3′ UTR between the normal termination codon (TC) and the first in-frame 3′ TC, and divided by the density of ribosomes in the coding sequence (CDS). G418 increased the RRTS of all of the UAA, UAG, and UGA TCs, whereas the suppressor tRNA^{Tyr} induced a much lower RRTS that is limited to the UAG TC (FIG. 9A). Even while the suppressor tRNAs induced lower readthroughs than G418, these tRNAs restored IDUA activity in fibroblasts at similar levels of efficiency G418 (FIG. 9B).

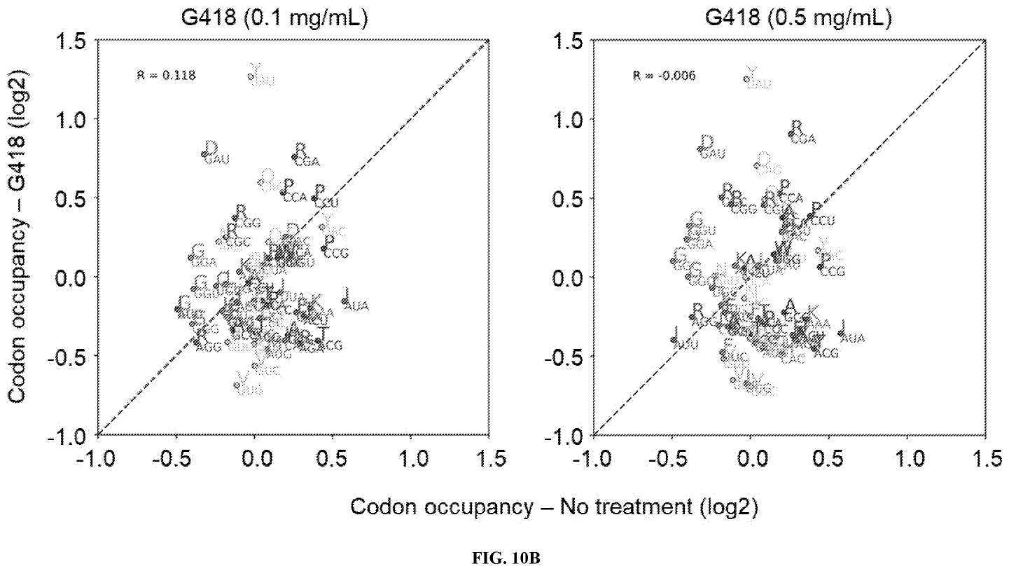

Additionally, the suppressor tRNAs caused less perturbation on translation elongation than G418. Ribosome profiling revealed minimal changes in codon occupancy by elongating ribosomes at transcriptome-wide coding sequences in Hurler patient fibroblasts that were treated with lentiviral vectors expressing EGFP and suppressor tRNA^{Tyr}, compared to control experiments (fibroblasts treated with a lentivirus vector expressing EGFP alone) (FIG. 10A). Conversely, ribosome profiling revealed changes in codon occupancy by elongating ribosomes at many codons in Hurler patient fibroblasts that were treated with lentiviral vectors expressing EGFP and suppressor tRNA^{Tyr}, compared to control experiments, suggesting that G418 causes global perturbation on translation elongation (FIG. 10B).

### SEQUENCES

In some embodiments a synthetic suppressor tRNA is encoded by a sequence comprising any one of SEQ ID NOs: 1-19, or a complement thereof. In some embodiments, a synthetic suppressor tRNA comprises the sequence set forth in any one of SEQ ID NOs: 20-38.

tRNA^{Tyr}(UAG):
SEQ ID NO: 1
CCTTCGATAGCTCAGTTGGTAGAGCGGAGGACTCTAGATCCTTAGGTCGCT
GGTTCGATTCCGGCTCGAAGGA
tRNA^{Leu}(UAG):
SEQ ID NO: 2
GGTAGCGTGGCCGAGCGGTCTAAGGCGCTGGATTCTAGCTCCAGTCTCTTC
GGGGGCGTGGGTTCAAATCCCACCGCTGCCA
tRNA^{Ser}(UAG):
SEQ ID NO: 3
GACGAGGTGGCCGAGTGGTTAAGGCGATGGACTCTAAATCCATTGTGCTCT
GCACACGTGGGTTCGAATCCCATCCTCGTCG
tRNA^{Lys}(UAG):
SEQ ID NO: 4
GCCTGGATAGCTCAGTTGGTAGAGCATCAGACTCTAAATCTGAGGGTCCAG
GGTTCAAGTCCCTGTTCAGGCG
tRNA^{Glu}(UAG):
SEQ ID NO: 5
TCCCACATGGTCTAGCGGTTAGGATTCCTGGTTCTAACCCAGGCGGCCCGG
GTTCGACTCCCGGTGTGGGAA
tRNA^{Gln}(UAG):
SEQ ID NO: 6
GGCCCCATGGTGTAATGGTTAGCACTCTGGACTCTAAATCCAGCGATCCGA
GTTCAAATCTCGGTGGGACCT
tRNA^{Trp}(UAG):
SEQ ID NO: 7
GGCCTCGTGGCGCAACGGTAGCGCGTCTGACTCTAGATCAGAAGGTTGCGT
GTTCAAATCACGTCGGGGTCA
tRNA^{Glu}(UAA):
SEQ ID NO: 8
TCCCACATGGTCTAGCGGTTAGGATTCCTGGTTTTAACCCAGGCGGCCCGG
GTTCGACTCCCGGTGTGGGAA
tRNA^{Gln}(UAA):
SEQ ID NO: 9
GGCCCCATGGTGTAATGGTTAGCACTCTGGACTTTAAATCCAGCGATCCGA
GTTCAAATCTCGGTGGGACCT
tRNA^{Tyr}(UAA):
SEQ ID NO: 10
CCTTCGATAGCTCAGTTGGTAGAGCGGAGGACTTTAGATCCTTAGGTCGCT
GGTTCGAATCCGGCTCGAAGGA
tRNA^{Leu}(UAA):
SEQ ID NO: 11
GTCAGGATGGCCGAGTGGTCTAAGGCGCCAGACTTTAGTTCTGGTCTCCAA
TGGAGGCGTGGGTTCGAATCCCACTTCTGACA
tRNA^{Ser}(UAA):
SEQ ID NO: 12
GTAGTCGTGGCCGAGTGGTTAAGGCGATGGACTTTAAATCCATTGGGGTTT
CCCCGCGCAGGTTCGAATCCTGCCGACTACG
tRNA^{Lys}(UAA):
SEQ ID NO: 13
GCCCGGCTAGCTCAGTCGGTAGAGCATGGGACTTTAAATCTCAGGGTCGTG
GGTTCGAGCCCCACGTTGGGCG
tRNA^{Trp}(UGA):
SEQ ID NO: 14
GGCCTCGTGGCGCAACGGTAGCGCGTCTGACTTCAGATCAGAAGGTTGCGT
GTTCAAATCACGTCGGGGTCA
tRNA^{Gly}(UGA):
SEQ ID NO: 15
GCGTTGGTGGTATAGTGGTTAGCATAGCTGCCTTCAAAGCAGTTGACCCGG
GTTCGATTCCCGGCCAACGCA
tRNA^{Arg}(UGA):
SEQ ID NO: 16
GGGCCAGTGGCGCAATGGATAACGCGTCTGACTTCAGATCAGAAGATTCTA
GGTTCGACTCCTGGCTGGCTCG
tRNA^{Cys}(UGA):
SEQ ID NO: 17
GGGGGTATAGCTCAGGGGTAGAGCATTTGACTTCAGATCAAGAGGTCCCCG
GTTCAAATCCGGGTGCCCCCT
tRNA^{Leu}(UGA):
SEQ ID NO: 18
ACCAGAATGGCCGAGTGGTTAAGGCGTTGGACTTCAGATCCAATGGATTCA
TATCCGCGTGGGTTCGAACCCCACTTCTGGTA
tRNA^{Ser}(UGA):
SEQ ID NO: 19
GTAGTCGTGGCCGAGTGGTTAAGGCGATGGACTTCAAATCCATTGGGGTTT
CCCCGCGCAGGTTCGAATCCTGTCGGCTACG
tRNA^{Tyr}(UAG):
SEQ ID NO: 20
CCUUCGAUAGCUCAGUUGGUAGAGCGGAGGACUCUAGAUCCUUAGGUCGCU
GGUUCGAUUCCGGCUCGAAGGA
tRNA^{Leu}(UAG):
SEQ ID NO: 21
GGUAGCGUGGCCGAGCGGUCUAAGGCGCUGGAUUCUAGCUCCAGUCUCUUC
GGGGGCGUGGGUUCAAAUCCCACCGCUGCCA
tRNA^{Ser}(UAG):
SEQ ID NO: 22
GACGAGGUGGCCGAGUGGUUAAGGCGAUGGACUCUAAAUCCAUUGUGCUCU
GCACACGUGGGUUCGAAUCCCAUCCUCGUCG
tRNA^{Lys}(UAG):
SEQ ID NO: 23
GCCUGGAUAGCUCAGUUGGUAGAGCAUCAGACUCUAAAUCUGAGGGUCCAG
GGUUCAAGUCCCUGUUCAGGCG
tRNA^{Glu}(UAG):
SEQ ID NO: 24
UCCCACAUGGUCUAGCGGUUAGGAUUCCUGGUUCUAACCCAGGCGGCCCGG
GUUCGACUCCCGGUGUGGGAA
tRNA^{Gln}(UAG):
SEQ ID NO: 25
GGCCCCAUGGUGUAAUGGUUAGCACUCUGGACUCUAAAUCCAGCGAUCCGA
GUUCAAAUCUCGGUGGGACCU
tRNA^{Trp}(UAG):
SEQ ID NO: 26
GGCCUCGUGGCGCAACGGUAGCGCGUCUGACUCUAGAUCAGAAGGUUGCGU
GUUCAAAUCACGUCGGGGUCA
tRNA^{Glu}(UAA):
SEQ ID NO: 27
UCCCACAUGGUCUAGCGGUUAGGAUUCCUGGUUUUAACCCAGGCGGCCCGG
GUUCGACUCCCGGUGUGGGAA
tRNA^{Gln}(UAA):
SEQ ID NO: 28
GGCCCCAUGGUGUAAUGGUUAGCACUCUGGACUUUAAAUCCAGCGAUCCGA
GUUCAAAUCUCGGUGGGACCU
tRNA^{Tyr}(UAA):
SEQ ID NO: 29
CCUUCGAUAGCUCAGUUGGUAGAGCGGAGGACUUUAGAUCCUUAGGUCGCU
GGUUCGAAUCCGGCUCGAAGGA
tRNA^{Leu}(UAA):
SEQ ID NO: 30
GUCAGGAUGGCCGAGUGGUCUAAGGCGCCAGACUUUAGUUCUGGUCUCCAA
UGGAGGCGUGGGUUCGAAUCCCACUUCUGACA
tRNA^{Ser}(UAA):
SEQ ID NO: 31
GUAGUCGUGGCCGAGUGGUUAAGGCGAUGGACUUUAAAUCCAUUGGGGUUU
CCCCGCGCAGGUUCGAAUCCUGCCGACUACG
tRNA^{Lys}(UAA):
SEQ ID NO: 32
GCCCGGCUAGCUCAGUCGGUAGAGCAUGGGACUUUAAAUCUCAGGGUCGUG
GGUUCGAGCCCCACGUUGGGCG
tRNA^{Trp}(UGA):
SEQ ID NO: 33
GGCCUCGUGGCGCAACGGUAGCGCGUCUGACUUCAGAUCAGAAGGUUGCGU
GUUCAAAUCACGUCGGGGUCA
tRNA^{Gly}(UGA):
SEQ ID NO: 34
GCGUUGGUGGUAUAGUGGUUAGCAUAGCUGCCUUCAAAGCAGUUGACCCGG
GUUCGAUUCCCGGCCAACGCA
tRNA^{Arg}(UGA):
SEQ ID NO: 35
GGGCCAGUGGCGCAAUGGAUAACGCGUCUGACUUCAGAUCAGAAGAUUCUA
GGUUCGACUCCUGGCUGGCUCG
tRNA^{Cys}(UGA):
SEQ ID NO: 36
GGGGGUAUAGCUCAGGGGUAGAGCAUUUGACUUCAGAUCAAGAGGUCCCCG
GUUCAAAUCCGGGUGCCCCCU
tRNA^{Leu}(UGA)
SEQ ID NO: 37
ACCAGAAUGGCCGAGUGGUUAAGGCGUUGGACUUCAGAUCCAAUGGAUUCA
UAUCCGCGUGGGUUCGAACCCCACUUCUGGUA
tRNA^{Ser}(UGA):
SEQ ID NO: 38
GUAGUCGUGGCCGAGUGGUUAAGGCGAUGGACUUCAAAUCCAUUGGGGUUU
CCCCGCGCAGGUUCGAAUCCUGUCGGCUACG

What is claimed is:

1. A method for treating a subject having a dominantly inherited disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

2. The method of claim 1, wherein the dominantly inherited disease is Huntington's disease, retinitis pigmentosa (RP), osteogenesis imperfecta, myotonic dystrophy, spinocerebellar ataxia 3, frontotemporal dementia (FTD), neurofibranatosis (type 1 or type 2), Marfan syndrome, Von Willebrand disease, familial hypercholesterolemia, tuberous sclerosis, amyotrophic lateral sclerosis (ALS), or Autosomal Dominant Polycystic Kidney Disease (ADPKD).

3. The method of claim 1 or 2, wherein the gene is HTT, RP1, RP2, COL1A1, COL1A2, DMPK, NF1, FBN1,VWF, TSC1, TSC2, SOD1, PKD1, or PKD2.

4. A method for treating a subject having a recessively inherited disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

5. The method of claim 4, wherein the recessively inherited diseases is cystic fibrosis (CF), Fanconi anemia, Pyruvate Dehydrogenase Deficiency, Pompe's disease, Gaucher's disease, phenylketonuria (PKU), or maple syrup urine disease (MSUD).

6. The method of claim 4 or 5, wherein the gene is CFTR, FANCA, PDHA1, GAA, GBA1, PAH, or BCKDHA.

7. A method for treating a subject having a mucopolysaccharide (MPS) storage disease, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

8. A method for treating a subject having a disease resulting from poor regulation of gene/protein expression, the method comprising administering to the subject a therapeutically effective amount of a synthetic suppressor transfer RNA (tRNA), wherein the synthetic suppressor tRNA comprises an anticodon region configured to recognize a nonsense mutation in a gene of the subject.

9. The method of claim 8, wherein the disease resulting from poor regulation of gene/protein expression is Rett Syndrome or CDKL5 deficiency disorder.

10. The method of claim 8 or 9, wherein the gene is MECP2 or CDKL5.

11. The method of any one of claims 1 to 10, wherein the nonsense mutation encodes a UAG, UAA, or UGA codon.

12. The method of any one of claims 1 to 11, wherein the anticodon region comprises a near-cognate sense codon.

13. The method of claim 12, wherein the near-cognate sense codon is selected from the group consisting of AAG, GAG, CAG, UGG, UCG, UUG, UAC, and UAU.

14. The method of any one of claims 1 to 13, wherein synthetic suppressor tRNA is charged with an amino acid selected from the group consisting of Lysine, Glutamic acid, Glutamine, Tryptophan, Serine, Leucine, and Tyrosine.

15. The method of any one of claims 1 to 14, wherein the synthetic suppressor tRNA is encoded by an expression construct comprising a nucleic acid sequence selected from the group consisting of SEQ ID NOs: 1-19.

16. The method of claim 15, wherein the nucleic acid sequence is operably linked to a promoter.

17. The method of claim 16, wherein the promoter is an RNA polymerase III promoter, optionally wherein the promoter is a U6 promoter.

18. The method of any one of claims 15 to 17, wherein the expression construct is flanked by viral terminal repeat sequences.

19. The method of claim 18, wherein the viral terminal repeat sequences are adeno-associated virus (AAV) inverted terminal repeats (ITRs).

20. The method of any one of claims 1 to 19, wherein the synthetic suppressor tRNA is encoded by a viral vector, optionally an rAAV vector or a lentiviral vector.

21. The method of claim 20, wherein the rAAV vector is encapsidated by AAV9 capsid proteins.

22. The method of any one of claims 7 or 11 to 21, wherein the MPS storage disease is Hurler syndrome (MPS I), Hurler-Scheie syndrome, Scheie syndrome, Hunter syndrome (MPS II), Sanfilippo syndrome A (MPS IIIA), Sanfilippo syndrome B (MPS IIIB), Sanfilippo syndrome C (MPS IIIC), Sanfilippo syndrome D (MPS IIID), Morquiro syndrome A (MPS IVA), Morquiro syndrome B (MPS IVB), Maroteaux-Lamy syndrome (MPS VI), Sly syndrome (MPS VII), or Natowicz syndrome (MPS IX).

23. The method of any one of claims 7 or 11 to 22, wherein the gene is selected from IDUA, IDS, SGSH, NAGLU, HGSNAT, GNS, GALNS, ARSB, GUSB, and HYAL1.

24. The method of any one of claims 1 to 23, wherein the synthetic suppressor transfer RNA (tRNA) is administered to the subject systemically, optionally wherein the administration is intravenous injection.

25. The method of any one of claims 1 to 24, wherein the administration of the synthetic suppressor transfer RNA (tRNA) results in read-through of the nonsense mutation in the gene of the subject.

26. The method of any one of claims 1 to 25, wherein the therapeutically effective amount results in an activity level of a protein expressed from the gene that is at least 0.5% of the activity level of a wild-type protein expressed by the gene.

27. The method of any one of claims 1 to 26, wherein the subject is a mammal, optionally wherein the subject is a human.

28. A method of increasing iduronidase activity in a cell, the method comprising administering to the cell a therapeutically effective amount of a synthetic suppressor transfer RNA (tRNA), wherein the synthetic suppressor tRNA comprises an anticodon region configured to recognize a nonsense mutation in an IDUA gene of the cell.

29. The method of claim 28, wherein the anticodon region comprises a near-cognate sense codon.

30. The method of claim 29, wherein the near-cognate sense codon is selected from the group consisting of AAG, GAG, CAG, UGG, UCG, UUG, UAC, and UAU.

31. The method of any one of claims 28 to 30, wherein the synthetic suppressor tRNA is charged with Tyrosine.

32. The method of any one of claims 28 to 31, wherein the synthetic suppressor tRNA is encoded by a viral vector.

33. The method of claim 32, wherein the viral vector is an rAAV vector.

34. The method of claim 33, wherein the rAAV vector is encapsidated by AAV9 capsid proteins.

35. A method of extended mRNA translation modulation in a cell, the method comprising delivering to the cell a viral vector encoding one or more synthetic suppressor transfer RNAs (tRNAs) configured to read-through certain stop codons of mRNAs in the cell.

36. The method of claim 35, wherein the viral vector is a rAAV vector.

37. The method of claim 35, wherein the viral vector is a lentiviral vector.

38. The method of any one of claims 35 to 37, wherein the one or more synthetic suppressor tRNAs induce fewer off-target readthroughs in the cell than an appropriate control cell to which is delivered aminoglycoside geneticin (G418) to stimulate read-through of the certain stop codons in the control cell.
